(19) United States
(12) Patent Application Publication
(10) Pub. No.: US 2001/0011070 A1
(43) Pub. Date: Aug. 2, 2001

(54) USE OF THREONINE FOR THE TREATMENT OF PHENYLKETONURIA

(76) Inventors: GILDA GEORGI, FRIEDRICHSDORF (DE); GUNTHER SAWATZKI, MUNZENBERG (DE)

Correspondence Address: BACON & THOMAS, 625 SLATERS LANE 4TH FLOOR, ALEXANDRIA, VA, 22314

(21) Appl. No.: 09/029,064

(22) PCT Filed: Sep. 18, 1996
(86) PCT No.: PCT/EP96/04093

§ 371 Date: None

(30) Foreign Application Priority Data
Sep. 18, 1995 (DE) 195 34 602.5

(51) Int. Cl. A61K 38/00; A01N 37/18

(52) U.S. Cl. 514/2

(57) ABSTRACT

The invention relates to the use of threonine for the treatment of phenylketonuria. The invention is based on the surprising discovery that the concentration of phenylalanine in the plasma of PKU patients can be reduced by additional doses of threonine. The object of the invention is therefore an composition for dietetic or pharmaceutical use in phenylketonuria, which composition contains at least 8 g threonine per 100 g amino acids. This composition may consist of foods, in particular formula foods and PKU dietetic products.

### DESCRIPTION

The invention relates to a composition for the dietetic or pharmaceutical treatment of phenyl-ketonuria and the use of threonine for the treatment of phenylketonuria.

Phenylketonuria (referred to below as PKU) is among the genetically determined diseases and is a metabolic disorder which if untreated usually leads to severe impairment of physical and mental development. Under normal nutritional conditions, phenylalanine is absorbed with all proteins of animal and plant origin. In persons with the disease, phenylalanine accumulates in the blood and tissues because of the metabolic disorder. The consequences of the sometimes excessively high phenylalanine level are metabolic imbalances, which can lead via various symptoms to irreversible, progressive mental disability.

PKU is not curable, but can be treated by appropriate diets, which are low in phenylalanine, so that the amount of phenylalanine introduced via the diet is only such that the synthesis of endogenous proteins for growth and regeneration is optimally ensured and at the same time the phenylalanine blood level lies in the normal range.

Since all proteins contain phenylalanine, these diets must be extremely low in protein. Hence with a low-phenylalanine diet, the PKU patients are supplied with too little of all other amino acids, which are just as important for life. Consequently, the other amino acids are supplied additionally in the form of a dietetic product which mostly contains free amino acids. Such a dietetic product is for example described in EP-A 0 488,078. A phenylalanine-free baby and infant food base is also described in EP-A 0 492,183. In this, there are also other statements concerning the PKU problem under discussion here. Further, other special dietetic products for the treatment of PKU are also explained there. Reference is hereby expressly made to the disclosure of these European patent applications.

The main disadvantage of the previously known phenylalanine-free amino acid mixtures and the dietetic products based thereon is their extremely foul taste, so that such phenylalanine-free dietetic products are often rejected by the patients who should preferably take these throughout their lives.

The purpose of the present invention is to provide an improved and simpler means for the dietary treatment or pharmaceutical treatment of phenylketonuria.

This purpose is accomplished by the teaching of the independent claims.

It has surprisingly been found that by administration of additional and in particular high doses of threonine the concentration of phenylalanine in the plasma of PKU patients can be reduced. Through the oral or parenteral administration of threonine, which leads to high threonine blood levels, the phenylalanine level in the blood can be reduced. This has for example the effect that with additional administration of threonine the diet of PKU patients does not have to be so strictly controlled as regards phenylalanine content. Moreover, as a result of the phenylalanine-lowering action of the threonine, more phenylalanine and thus also more protein can be supplied to PKU patients in the diet, without this leading to damage to health. This has the great advantage that significantly more foods are suitable for the PKU patients. At the same time, the previously necessary daily supply of free amino acids can be correspondingly reduced.

In the context of the present documents, if the term “additional” is mentioned in connection with the amount of threonine, this means that a patient is supplied with or administered an amount of threonine which is higher or greater than that amount which a PKU patient previously ingested, for example with the usual diet including usual PKU supplements, or which a PKU patient should ingest on the basis of previous recommendations.

The additional threonine can be administered to a PKU patient in any suitable form. Thus it is for example possible to treat normal foods with threonine and thereby to supply the patient with an increased amount of threonine. Furthermore, it is possible to supply the increased threonine input in the form of a food and in particular a formula food. Administration in the form of a pharmaceutical is also possible.

Such foods, etc., include for example baby milks, childrens' foods, foods for adolescents and adults, dietetic products and food supplements.

According to a preferred embodiment, the composition according to the invention is in the form of a dietetic foodstuff or a food base, which contains the threonine in an amount of 8 to 80 g per 100 g amino acids. Here the choice of the amount of threonine depends on which other foods and/or food supplements, for example previously known food supplements, are made available to the patient.

According to a further preferred embodiment, the composition according to the invention is in the form of a pharmaceutical composition or a food supplement in which the threonine makes up 8 to 100 g per 100 g amino acids. Thus in the simplest case, a patient can be provided with a pharmaceutical composition or a food supplement which contains exclusively threonine as the active component. Thus it is for example possible to administer normal capsules containing pure threonine (free amino acid). By administration of such free threonine or threonine capsules, the threonine content of foods, food supplements and PKU dietetic products can be raised, without changing the other amino acids and/or proteins in their composition.

The threonine can be incorporated in the individual foods, food supplements, preparations and pharmaceutical compositions in the form of the free amino acid. Moreover, the threonine can be present in the form of its salts, in the form of threonine-containing peptides (for example bovine glycomacropeptide), in the form of threonine-rich proteins and hydrolysates thereof and also in the form of mixtures of these threonine sources.

The same applies for the other amino acids, which can be in any suitable form, for example as peptides, as proteins and as free amino acids or as a mixture thereof.

However in the context of the present documents, if a certain amount of threonine per 100 g amino acids is mentioned, then the values for the free amino acids are used in the calculations, even if these amino acids are present in bound form and thus as peptide or protein. Thus the quantity information for the threonine and for the amino acids is based on the free acids.

Thus the core of the present invention consists in that additional amounts of threonine are administered to a PKU patient, in order in this way to decrease the phenylalanine blood level. The manner of the threonine administration here is of lesser importance and should be chosen on the basis of the needs of the patient, the requirements of the dietary plan or taking account of other criteria important for the patient.

Thus the only decisive factor is that a PKU patient ingests an additional amount of threonine in any manner. According to a preferred embodiment, a PKU patient ingests at least an amount of threonine such that the amount of threonine supplied to the body makes up at least 8 g per 100 g of the total amount of amino acids supplied to the body. In the context of the present application, if a threonine quantity range of 8 g to 100 g or of 8 g to 80 g is mentioned, then all values lying between these are thereby disclosed, for example 8, 9, 10, 11, 12, . . . 15, 16, . . . 30, 31, . . . 45, 46, 47, 48, 49, 50 . . . 60, 61, . . . , 70, 71, 72, . . . , 80, 81, . . . , 90, 91, . . . 100.

In the simplest case, therefore, a PKU patient can ingest a normal food. In order now to ingest the necessary amount of threonine, this patient can take the threonine as free acid, which is for example incorporated in a capsule.

Also an object of the invention is a procedure for the treatment of PKU wherein an additional amount of threonine is administered to a PKU patient as well as the amount of threonine which the PKU patient usually or normally ingests.

However the main field of application of the invention consists in that it is possible to replace a part of the PKU foods already known and present on the market, generally very foul-tasting, with threonine-rich products or threonine in free form. Through this threonine, the phenylalanine level in the patient's blood is reduced, so that increased use can also be made of conventional foods, such as baby milks, purees, milk, milk products, fruit, vegetables, etc., in the diets.

The invention is explained below in more detail on the basis of the following examples:

### EXAMPLE 1

### Baby Milk for Babies from 0 to 3 Months (daily amount)

The starting basis is a normal PKU diet for this age, for example the composition Milupa PKU 1 for babies. 100 g PKU 1 contain 60.3 g amino acids (overall: corresponds to about 50.3 g protein), 0.0 g fat, 21.0 g carbohydrates and 15.8 [sic] minerals and trace elements, and the remainder water.

50% of this composition are treated with a quantity of glycomacropeptide and/or of a normal commercial milk baby food (for example Milumil 1) corresponding to the protein content. Thereby, an amount of glycomacropeptide and/or of a normal commercial milk baby food is added such that the protein content of this added amount of glycomacropeptide and/or of a normal commercial milk baby food corresponds to the protein content of the replaced part of the PKU diet. In addition, the different protein sources are mixed in such a way that 2.5 to 3.8 g protein come from the PKU diet, 2.5 to 3.8 g protein from the glycomacropeptide and 3.8 to 4.2 g protein from the normal commercial milk baby food. The quantity of threonine contained in this mixture is 1.0 to 1.2 g and is higher by a factor of 3.3 [sic] to 2.4 than the recommended minimum quantity of threonine for healthy children.

### EXAMPLE 2

### Diet for Children from 1 to 3 Years (daily amount)

The starting basis is a normal PKU diet for this age, for example the composition Milupa PKU 2 for infants and school-children. 100 g PKU 2 contain 80.1 g amino acids (overall: corresponds to 66.8 g protein), 0.0 g fat, 8.5 g carbohydrates and 7.7 g minerals and trace elements (remainder water). 25% of this phenylalanine-free PKU diet are replaced with a quantity of normal commercial finished purees, fruit and vegetables corresponding to the protein content. In addition, the protein sources are mixed in such a manner that 10.1 g protein of the finished diet come from the normal PKU diet and 6.0 g protein from the finished purees, fruit and/or vegetables.

At the same time, 4 g threonine in free form are administered daily in the form of capsules to the children taking this diet.

The quantity of threonine contained in this combination of diet and threonine in capsule form amounts to 4.9 g and is higher by a factor of 5.4 than the minimum recommended daily amount of threonine for healthy children.

### EXAMPLE 3

### Diet for School Children from 7 to 10 Years (daily amount)

The starting basis is the PKU diet described in Example 2. 70% of this phenylalanine-free PKU diet are replaced with a mixture of glycomacropeptide and normal commercial milk products, fruit and vegetables, etc., corresponding to the protein content. In addition, the protein sources are mixed in such a manner that 6.7 g protein of the finished diet come from the PKU diet used at the start, 12.3 g protein from the glycomacropeptide and 8.2 g protein from the normal commercial milk products, fruit, vegetables, etc.

At the same time, 7 g threonine, which can be in the form of capsules or coated tablets or are incorporated in an instant drink, are administered to the treated school children.

The overall quantity of threonine administered to the school children in this combination amounts to 10.1 g and is higher by a factor of 10.1 than the minimum recommended daily amount of threonine for healthy children.

1. Composition for the dietetic or pharmaceutical treatment of phenylketonuria,
characterised by
a content of at least 8 g threonine per 100 g amino acids.

2. Composition according to claim 1,
characterised in that
the threonine is present as free amino acid, as salt, as threonine-containing peptide (in particular as bovine glycomacropeptide), as threonine-rich protein or as protein hydrolysate or as a mixture thereof.

3. Composition according to claim 1 or 2 in the form of a dietetic food of a PKU diet, or a food base,
characterised in that
it contains threonine in an amount of 8 to 80 g per 100 g amino acids.

4. Composition according to claim 1 or 2,
characterised in that
it is present in the form of a pharmaceutical composition or a food supplement.

5. Composition according to claim 1, in the form of a pharmaceutical composition or a food supplement,
characterised in that
as active component it contains exclusively threonine, in particular as free amino acid.

6. Use of threonine for the treatment of phenylketonuria or for lowering the phenyl-alanine level in the plasma.

7. Use according to claim 6,
characterised in that
the threonine is used in an amount of at least 8 g per 100 g amino acids.

8. Process for the treatment of PKU, wherein an additional amount of threonine is administered to a PKU patient, as well as the amount of threonine which the PKU patient usually or normally ingests.
